(19) United States
(12) Patent Application Publication
(10) Pub. No.: US 2024/0033080 A1
(43) Pub. Date: Feb. 1, 2024

(54) PROSTHETIC HEART VALVE DELIVERY ASSEMBLIES WITH MULTIPLE LOCATION PRESSURE SENSING

(71) Applicant: EDWARDS LIFESCIENCES CORPORATION, Irvine, CA (US)

(72) Inventors: Nicholas Scott Steenwyk, Minneapolis, MN (US); Gil Senesh, Adi (IL)

(21) Appl. No.: 18/486,443

(22) Filed: Oct. 13, 2023

Related U.S. Application Data
(63) Continuation of application No. PCT/US2022/026740, filed on Apr. 28, 2022.
(60) Provisional application No. 63/181,722, filed on Apr. 29, 2021.

(51) Int. Cl. A61F 2/24; A61B 5/00; A61B 5/0215

(52) U.S. Cl. CPC A61F 2/2433; A61B 5/6851; A61B 5/02158; A61F 2250/0098; A61M 2025/0002

(57) ABSTRACT

A multiple location pressure sensing device for use with a prosthetic heart valve delivery assembly is disclosed in several examples. As one example, a prosthetic heart valve delivery assembly with a multiple location pressure sensing device can include a delivery apparatus, a guidewire, and a delivery apparatus. The example also includes a first pressure sensor configured to be positioned near an inlet end of a prosthetic heart valve and a second pressure sensor configured to be positioned near an outlet end of the prosthetic heart valve, wherein the first and second sensor are configured to measure a pressure gradient across the prosthetic heart valve.

### CROSS-REFERENCE TO RELATED APPLICATIONS

This application is a continuation of International Patent Application No. PCT/US2022/026740, filed Apr. 28, 2022, which claims the benefit of U.S. Provisional Patent Application No. 63/181,722 filed on Apr. 29, 2021. The prior applications are incorporated by reference herein in their entirety.

### FIELD

The present disclosure relates generally to delivery assemblies for transcatheter prosthetic heart valves and, more particularly, to delivery assemblies having multiple location pressure sensing, as well as methods for using the same.

### BACKGROUND

Prosthetic heart valves have been used for many years to treat cardiac valvular disorders. The native heart valves (such as the aortic, pulmonary, tricuspid, and mitral valves) serve critical functions in assuring the forward flow of an adequate supply of blood through the cardiovascular system. These heart valves can be rendered less effective by congenital, inflammatory, or infectious conditions. Such conditions can eventually lead to serious cardiovascular compromise or death. For many years the definitive treatment for such disorders was the surgical repair or replacement of the valve during open heart surgery, but such surgeries are dangerous and prone to complication.

More recently a transvascular technique has been developed for introducing and implanting a radially-expandable prosthetic heart valve to replace a defective native heart valve using a flexible catheter in a manner that is less invasive than open heart surgery. In this technique, a radially expandable prosthetic valve is mounted in a crimped or radially-compressed state on the end portion of a flexible delivery apparatus and advanced through a blood vessel of the patient until the valve reaches the implantation site. The prosthetic valve is then expanded to its functional size at the site of the defective native valve, such as by inflating a balloon on which the valve is mounted, for example by injecting saline into the balloon. Once the prosthetic valve is in place, the balloon is deflated, and the delivery apparatus is withdrawn.

As an alternative to balloon-expandable prosthetic valves, the prosthetic valve can have a resilient, self-expanding stent or frame that expands the valve to its functional size when it is advanced from a delivery apparatus at the distal end of the delivery sheath and/or the guidewire. As another alternative, a prosthetic valve can be mechanically expandable via one or more actuators.

After the installation of a prosthetic heart valve, it is important to determine that the valve has been correctly installed and/or desirably positioned. As such, there is a need for devices and methods for monitoring the functionality of the prosthetic valve and/or other indicators to ensure that prosthetic valve has been correctly installed and/or desirably positioned.

### SUMMARY

Disclosed herein are delivery assemblies for prosthetic heart valves having pressure sensors configured to simultaneously measure pressure at two or more locations. The delivery assemblies can be used to measure the pressure gradient across a prosthetic heart valve after it has been installed in the native heart valve of a patient, without the need to insert any additional instrumentation into the patient having a prosthetic heart valve installed. In some examples, the delivery assemblies may comprise a delivery apparatus and a guidewire. In other examples, the delivery assemblies may include only a delivery apparatus or only a guidewire. Also disclosed herein are methods for using the delivery assemblies disclosed.

Certain examples of the disclosure concern a delivery assembly for a prosthetic heart valve, having a delivery apparatus and a guidewire extending through the delivery apparatus. The example also includes a first pressure sensor configured to be positioned near an inlet end of a prosthetic heart valve and a second pressure sensor configured to be positioned near an outlet end of the prosthetic heart valve. The first pressure sensor and second pressure sensor are positioned on the delivery apparatus or the guidewire and are configured to measure a pressure gradient across the prosthetic heart valve.

Certain examples of the disclosure concern a method of measuring a pressure gradient across a prosthetic heart valve, including placing a first pressure sensor at a first sensor location near an inlet end of a prosthetic heart valve installed in a heart of a patient and placing a second pressure sensor at a second sensor location near an outlet end of the prosthetic heart valve installed in the patient. The method also includes simultaneously measuring a first pressure at the first sensor location and a second pressure at the second sensor location and calculating the pressure gradient across the prosthetic heart valve from the first pressure measured at the first sensor location and the second pressure measured at the second sensor location.

Certain examples of the disclosure concern another method of measuring a pressure gradient across a prosthetic heart valve, including deploying an assembly having a delivery apparatus, a guidewire, a radially expandable prosthetic heart valve, and at least two pressure sensors into a heart of a patient. The method also includes expanding the prosthetic heart valve into a native heart valve of the patient, positioning a first pressure sensor at a first location in front of an inlet of the prosthetic heart valve in a direction of flow, and positioning a second pressure sensor at a second location after an outlet of the prosthetic heart valve in the direction of flow. The method also includes simultaneously measuring a first pressure at the first location of the first pressure sensor and a measuring second pressure at the second location of the second pressure sensor and calculating the pressure gradient across the prosthetic heart valve.

Certain examples of the disclosure concern another delivery assembly for a prosthetic heart valve, having a delivery apparatus, a first pressure sensor configured to be positioned near an inlet end of a prosthetic heart valve, and a second pressure sensor configured to be positioned near an outlet end of the prosthetic heart valve. The first pressure sensor and second pressure sensor are positioned on the delivery apparatus and are configured to measure a pressure gradient across the prosthetic heart valve.

Certain examples of the disclosure concern another delivery assembly for a prosthetic heart valve, comprising a guidewire, a first pressure sensor configured to be positioned near an inlet end of a prosthetic heart valve, and a second pressure sensor configured to be positioned near an outlet end of the prosthetic heart valve. The first pressure sensor and second pressure sensor are positioned on the guidewire and are configured to measure a pressure gradient across the prosthetic heart valve.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve in a radially compressed condition and an inflatable balloon in a deflated condition.

FIG. 2 is a side view of a prosthetic heart valve in a radially expanded condition and an inflated balloon in and inflated condition.

FIG. 3 is a side view of a prosthetic heart valve delivery assembly positioned in the heart of a patient before the installation procedure.

FIG. 4 is a side view of a prosthetic heart valve delivery assembly positioned in the heart of a patient during the installation procedure.

FIG. 5 is a side view of a prosthetic heart valve delivery assembly positioned in the heart of a patient after the installation procedure.

FIG. 6 is a side view of a prosthetic heart valve delivery assembly with a multiple location pressure sensing device having both sensors on a guidewire.

FIG. 7 is a side view of a prosthetic heart valve delivery assembly with a multiple location pressure sensing device having one sensor on a nosecone and the other on a guidewire or delivery sheath.

FIG. 8 is a side view of a prosthetic heart valve delivery assembly with a multiple location pressure sensing device having one sensor on a nosecone and the other on a nosecone shaft.

FIG. 9 is a side view of a prosthetic heart valve delivery assembly with a multiple location pressure sensing device having one sensor on a guidewire and the other on a nosecone shaft.

FIG. 10 is a side view of a prosthetic heart valve delivery assembly with a multiple location pressure sensing device having one sensor on a guidewire and the other on a nosecone.

FIG. 11 is a schematic view of a guidewire having a recessed pressure sensor and a radiopaque marker.

FIG. 12 is a perspective view of a guidewire having a recessed pressure sensor.

FIG. 13 is a schematic view of an inflatable balloon having a plurality of pressure sensors with an external display.

FIG. 14 is a perspective view of a prosthetic heart valve according to one example.

FIG. 15 is a perspective view of a prosthetic heart valve frame according to another example, shown in the radially compressed condition.

FIG. 16 is a perspective view of the prosthetic heart valve frame of FIG. 16 in the radially expanded condition.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic heart valve assembly and implantation and structures of the prosthetic heart valve, “proximal” refers to a position, direction, or portion of a component that is closer to the user and a handle of the delivery assembly or apparatus that is outside the patient, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic heart valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or.”

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

Overview of Disclosed Technology

The pressure gradient across the prosthetic heart valve is a key metric for evaluating whether a valve has been correctly and safely installed and/or for predicting the future performance of the valve. The pressure gradient across the prosthetic heart valve is defined as the difference between the blood pressure at the inlet end of a the prosthetic heart valve and the blood pressure at the outlet end of the prosthetic heart valve, and can be measured while the heart is in ventricular systole. While the present disclosure chiefly discusses measurement of the pressure gradient across the prosthetic heart valve while the heart is in ventricular systole, it is to be understood that measurements may be taken at other points of interest, such as when the heart is in ventricular diastole.

When the pressure gradient across a prosthetic heart valves is too high, it may indicate that the prosthetic heart valve is insufficiently expanded, and that the stress being put on the prosthetic heart valve in the patient's body is dangerously high. A pressure gradient that exceeds safe pressure levels can cause, for example, damage to the leaflets or to the frame of the prosthetic heart valve, or may cause the prosthetic heart valve to become dislodged or dislocated within the patient's native heart valve at some point after the installation procedure has been completed. Such damage or dislocation to the prosthetic heart valve can cause serious complications, including leaky flow, heart damage or even patient death. A pressure gradient that is too low (or negative) may indicate an improper installation that has resulted in backflow.

Another concern with the measurement of the pressure gradient across an installed prosthetic heart valve is the difficulty with ensuring that the measurements accurately capture an accurate pressure gradient at the time of measurement. Currently, pressure gradient is most frequently measured using Doppler ultrasound or one or more diagnostic catheters. Doppler ultrasound is commonly used since it is non-invasive and can be repeated after the procedure. But Doppler ultrasound can be difficult to use (especially for heavier patients), requires an echo operator, and has high variability. Additionally, Doppler ultrasound measurements do not capture simultaneous pressure measurements, because the pressure on one side of the prosthetic heart valve and the pressure on the other side of the prosthetic heart valve must be measured at different times, which can cause inaccuracies in the calculated pressure gradient.

A single diagnostic catheter or pressure guidewire is an alternative to Doppler ultrasound that can be used to measure pressure directly in a first part of the heart such as the ventricle and then be pulled back to measure pressure in a second part of the heart such as the aorta, but this method does not allow simultaneous measurements in both the aorta and the ventricle. In the case of a catheter, placing a catheter across the valve may make the pressure measurement less accurate.

As an alternative approach, two diagnostic catheters may be introduced to measure pressure-one positioned in a first part of the heart such as the ventricle and one positioned in a second part of the heart such as the aorta, for example. This allows simultaneous measurement across an installed prosthetic heart valve. However, this approach introduces a second device into the heart which can impact pressure measurement and cause additional difficulty and complexity in the installation procedure for the prosthetic heart valve.

Examples of pressure guidewires using a single pressure sensor include, for example, Opsen's “Optowire,” the “Omniwire” of Koninklijke Philips N.V., and Boston Scientific's “Comet II Pressure Guidewire.” Abbott's “Pressurewire™ X Guidewire” is an example of a wireless pressure guidewire.

Patents describing pressure guidewires include, for example, U.S. Pat. No. 12,702,162 assigned to Opsens Inc., U.S. Pat. No. 12,499,820 assigned to Boston Scientific Scimed Inc., and U.S. Pat. No. 12,702,170 assigned to Zurich Medical Corp., all of which are hereby incorporated by reference herein. Related technology may be found in, for example, U.S. Pat. Nos. 7,259,862, 7,689,71, and 8,752,435, all of which are hereby incorporated by reference herein.

There is therefore a need for a low-profile, singular device or system to take direct pressure measurements on either side of an installed prosthetic heart valve simultaneously.

Disclosed herein are multiple location pressure sensing devices for simultaneous measurement of pressure on either side of a prosthetic heart valve installed in a patient. Multiple location pressure sensing devices generally comprise two or more pressure sensors. The two or more pressure sensors are configured for use with a prosthetic heart valve delivery assembly and can be positioned on either side of an installed prosthetic heart valve. Such prosthetic heart valve delivery assemblies can include a guidewire, and a delivery apparatus. Certain alternative examples of the prosthetic heart valve delivery assemblies disclosed herein may comprise only a guidewire or only a and a delivery apparatus, that is either the delivery apparatus or the guidewire may be omitted. In particular examples, the two or more sensors may be independently positionable to allow a physician to precisely control how far from the prosthetic heart valve pressure measurements are taken.

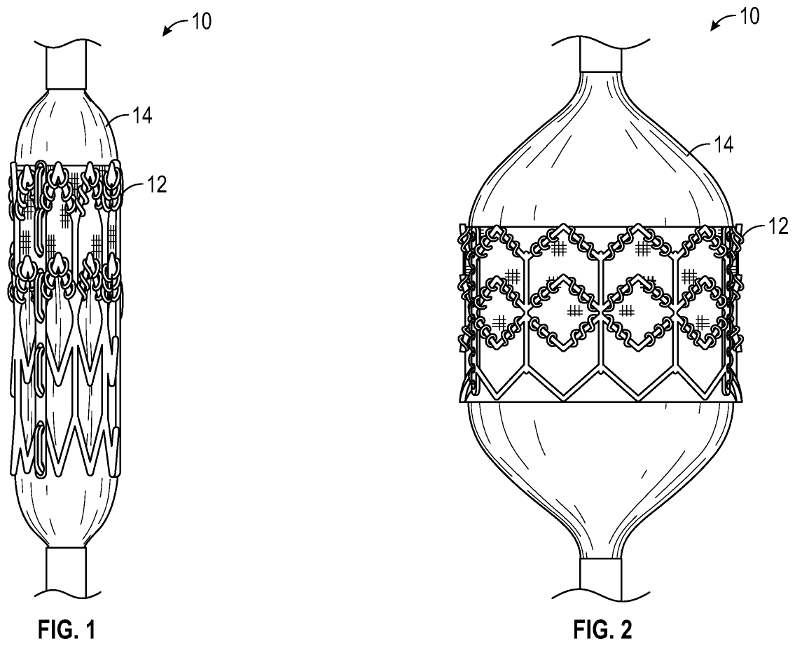

Prosthetic heart valves for use with the multiple may be configured to be radially expandable from a compressed configuration, as illustrated in FIGS. 1-2. For example, a prosthetic heart valve 12 may be positioned for installation while in a crimped or radially compressed configuration, as shown in FIG. 1. Once the prosthetic heart valve 12 is properly positioned, it may be expanded, for example by inflation of a balloon 14 until it is in an expanded state, as shown in FIG. 2.

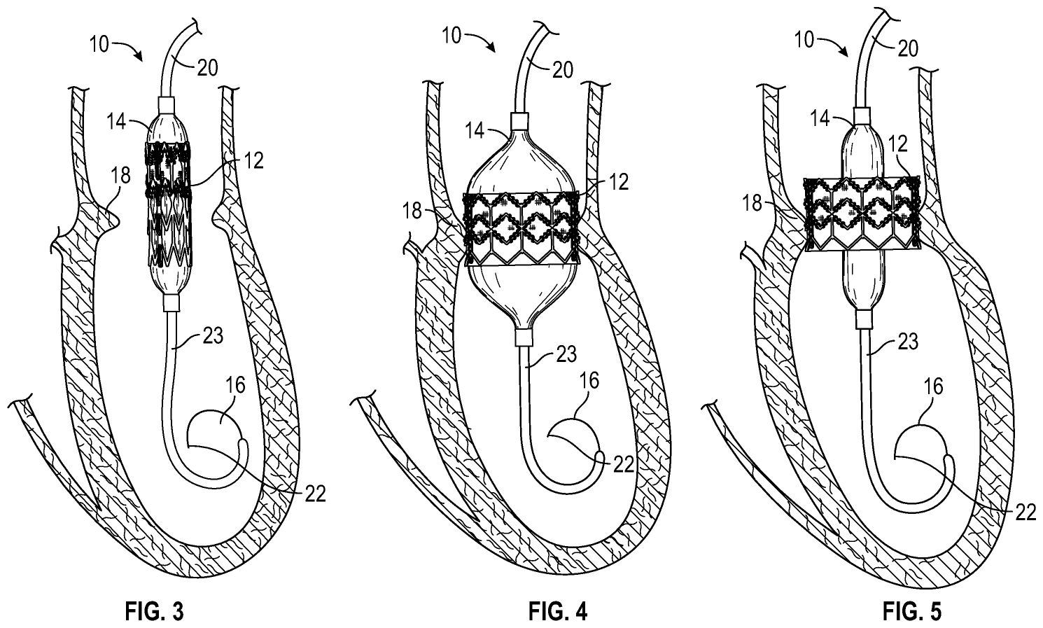

FIGS. 3-5 show an exemplary prosthetic heart valve installation procedure in which a multiple location pressure sensing device might be used. A prosthetic heart valve delivery assembly may be guided to a position within the heart of the patient wherein a guidewire 16 passes through the native heart valve 18. A delivery apparatus 20 and a prosthetic heart valve may be passed along the guidewire 16 until the prosthetic heart valve 12 is positioned within the native heart valve 18, as shown in FIG. 3. A balloon 14 may then be inflated to expand the prosthetic heart valve 12 until the outer diameter of the prosthetic heart valve 12 conforms to the inner diameter of the native heart valve 18, as shown in FIG. 4. When the prosthetic heart valve 12 has been expanded to the desired diameter, the balloon 14 may then be deflated as shown in FIG. 5 for removal from installation location.

After the prosthetic heart valve has been expanded to fit the native heart valve, it may cause resistance to the flow of blood through the heart, which in turn may result in a pressure gradient across the installed prosthetic heart valve. If the pressure gradient across a prosthetic heart valve is too high, it may cause complications, such as damage to the leaflets or to the frame of the prosthetic heart valve, or may cause the prosthetic heart valve to become dislodged or dislocated within the patient's native heart valve at some point after the installation procedure has been completed. Such damage or dislocation to the prosthetic heart valve can cause serious complications, including leaky flow, heart damage or even patient death. For this reason, a multiple location pressure sensing device, such as examples disclosed herein may be used to measure the pressure gradient across the prosthetic heart valve, to determine whether the pressure gradient is within acceptable limits.

Examples of the prosthetic heart valve delivery assemblies incorporating a multiple location pressure sensing device disclosed herein may be used to facilitate the safe replacement of any of the native valves in a patient's heart. For example, such assemblies may be used in the replacement of a native aortic valve and be configured to measure a pressure gradient between the left ventricle and the aorta. Alternatively, such assemblies may be used in the replacement of a native mitral valve and may be configured to measure the pressure gradient between the left atrium and the left ventricle. In another application, such assemblies may be used in the replacement of a native tricuspid valve and be configured to measure the pressure gradient between the right atrium and right ventricle. In yet another application, such assemblies may be used in the replacement of a native pulmonary valve and be configured to measure the pressure gradient between the right ventricle and the pulmonary artery.

Examples of Disclosed Technology

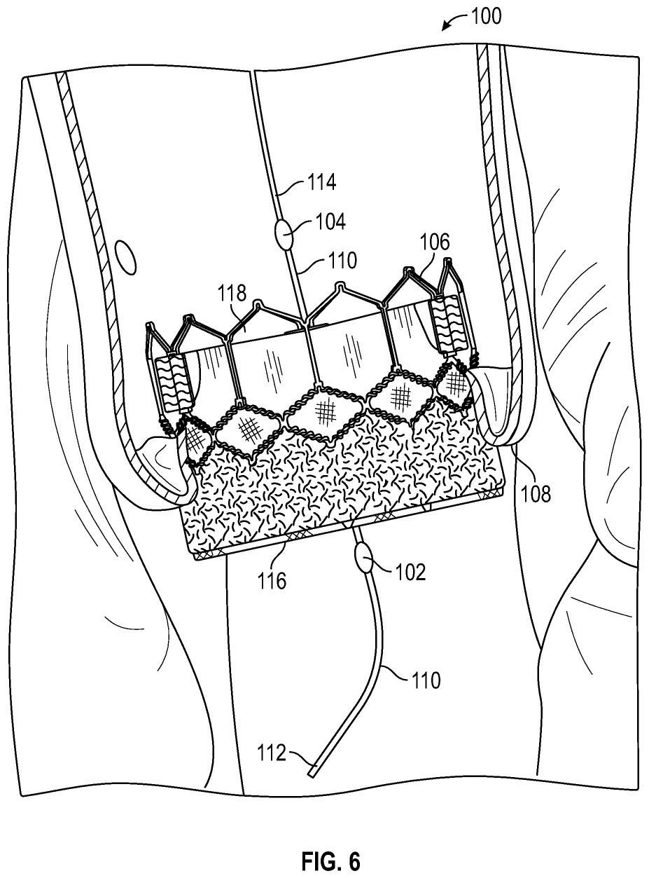

In a general example, a prosthetic heart valve delivery assembly 100 incorporating a multiple location pressure sensing device according to the present disclosure comprises at least a first pressure sensor and a second pressure sensor configured to be positioned on either end of an installed prosthetic heart valve, a data transmission mechanism, and an external display mechanism. In one particular example illustrated in FIG. 6, the multiple location pressure sensing device may comprise a distal sensor 102, a proximal sensor 104, a data transmission mechanism and an external display. The distal sensor 102 and the proximal sensor 104 may be configured to be positioned on either end of an installed prosthetic heart valve 106 after it has been installed in native heart valve 108. The distal sensor 102 and the proximal sensor 104 may measure pressure at locations distal to and proximal from the installed prosthetic heart valve 106 and may be configured to calculate a pressure gradient across the prosthetic heart valve 106 thereby. The distal sensor 102 and the proximal sensor 104 may be in communication with an external display or readout, through a data communication mechanism. Suitable sensors may include optical pressure sensors, piezoelectric pressure sensors, strain gauge sensors, and/or any combination thereof.

Background information on optical pressure sensors, such as Fabry Perot sensors for example, may be found in “Pressure Sensors: The Design Engineer's Guide” (Avnet Abacus, 2020), at https://www.avnet.com/wps/portal/abacus/solutions/technologies/sensors/pressure-sensors/core-technologies/optical/, which is incorporated by reference herein. In some examples of the present disclosure, the design allows blood flow to run parallel to the sensor so as not to distort the pressure measurement.

In some examples, recent advances in sensor technology may be incorporated. As examples, recent advances in sensor technology are presented in research articles entitled, “Epidermal Electronics for Noninvasive, Wireless, Quantitative Assessment of Ventricular Shunt Function in Patients with Hydrocephalus” (Krishnan et al., Science Translational Medicine 31 Oct. 2018: Vol. 12, Issue 465, eaat8437) and “Continuous, Noninvasive Wireless Monitoring of Flow of Cerebrospinal Fluid Through Shunts in Patients with Hydrocephalus” (Krishnan et al., NPJ Digit Med. 2020; 3: 29, Published online 2020 Mar. 6), both of which are incorporated by reference herein. Krishnan et al. fabricated thin, soft, flexible, skin-conformal, epidermally adherent sensors to monitor a subdermal ventricular catheter (shunt) function. The sensors detected shunt malfunctions in patients that were confirmed by imaging or surgery.

Multiple location pressure sensing devices disclosed herein can be configured for use with a prosthetic heart valve delivery assembly having a guidewire, a delivery apparatus, and a prosthetic heart valve. In some examples, the prosthetic heart valve delivery assembly further comprises an inflatable balloon configured to expand the prosthetic heart valve from a crimped or radially-collapsed state to an expanded state. In one general example illustrated in FIGS. 3-5, a prosthetic heart valve delivery assembly 10 may comprise a prosthetic heart valve 12, a guidewire 16, and a delivery apparatus 20. Guidewire 16 has a distal end 22 and a proximal end and can be configured to pass through the cardiovascular system of a patient during a prosthetic heart valve installation operation, until reaching the intended installation location of a radially-expandable prosthetic heart valve 12, such as native heart valve 18.

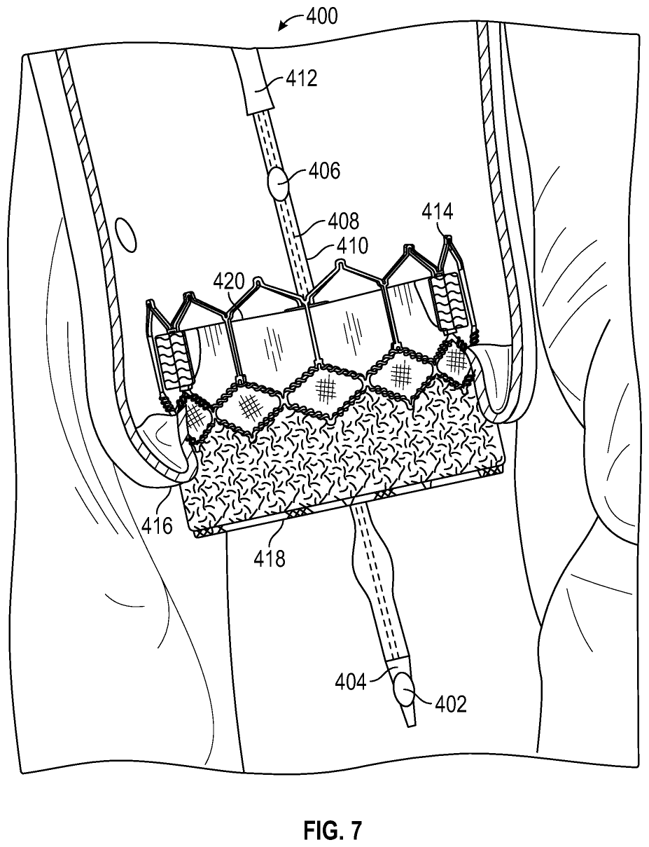

In one general example, the delivery apparatus 20 is configured to travel along guidewire 16 and carry a prosthetic heart valve 12 in a crimped or radially compressed condition towards the distal end 22 of the guidewire 16. In some examples, the delivery apparatus may also carry a balloon 14 and/or a nosecone and a nosecone shaft as shown in FIGS. 7-10. The multiple location pressure sensing apparatus examples disclosed herein may be incorporated in prosthetic heart valve delivery assemblies comprising a guidewire. As best illustrated in FIG. 6 a guidewire 110 may be designed to extend through the cardiovascular system of the patient, and pass through the native heart valve 108 to be replaced. The guidewire 110 may be configured to guide a delivery apparatus, as discussed in greater detail below, as well as the prosthetic heart valve 106 to be installed in the native heart valve 108. Other components of the delivery assembly may be configured to travel along the guidewire from the proximal end to the distal end 112 of the guidewire 110, or from the distal end 112 to the proximal end.

In some examples, a pressure guidewire further comprises a cable for the transmission of data, with the cable running through the center of the guidewire to transmit data such as pressure data from the pressure sensor to an external monitor. In one preferred example, the cable may be a fiberoptic cable, however it is to be understood that other cables suitable for data transmission, such as electrical cables may be used instead of fiberoptic cables. Data from multiple optical pressure sensors may be transmitted in this manner although, as will be discussed, other methods of data transmission such as wireless systems may be employed.

Concerning guidewires, it is to be understood that pressure guidewires may have various features. A few non-limiting examples of pressure guidewires, related pressure sensors, housings and/or sensor windows may be found, for example, in U.S. Pat. Nos. 12,702,162, 12,499,820, and 12,702,170, all of which are hereby incorporated by reference herein.

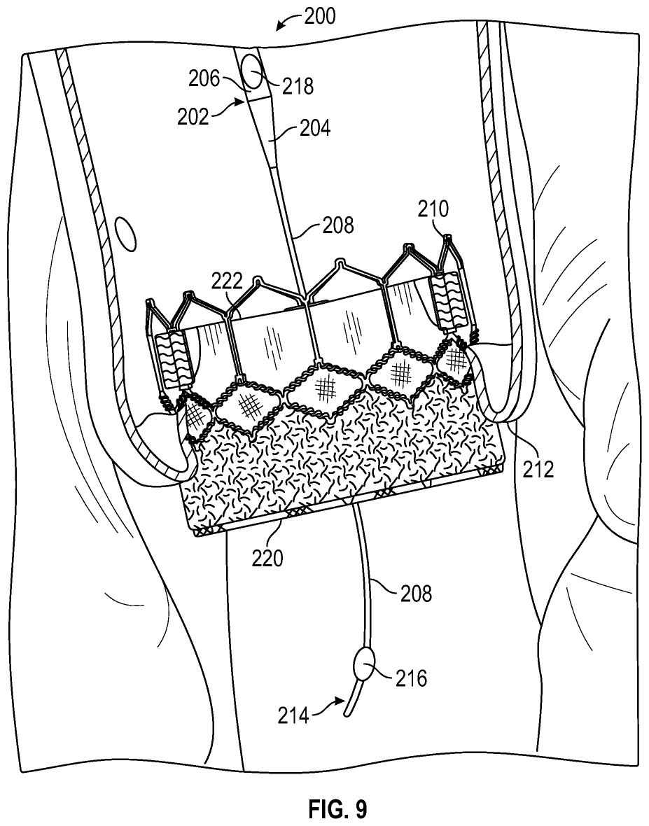

Turning now to FIGS. 6-10, the multiple location pressure sensing devices disclosed herein can be configured for use with various examples of a prosthetic heart valve delivery system further comprising a delivery apparatus. An example of a prosthetic heart valve delivery assembly 200 having a delivery apparatus 202 is shown in FIG. 9. As illustrated in FIG. 9, the delivery apparatus can further consist of a nosecone 204 (sometimes called a guide cone) and a nosecone shaft 206 (sometimes called a pusher or pushing element). The delivery apparatus 202 is configured to pass along the length of a guidewire 208 and to carry a prosthetic heart valve 210 to an installation site, such as a native heart valve 212. The nosecone 204 can be positioned towards the front end or distal end of the delivery apparatus 202 relative to the nosecone shaft 206, with the nosecone shaft 206 positioned behind the nosecone 204. In certain examples, the nosecone 204 and the nosecone shaft 206 are separate pieces, configured to be moved either in tandem or separately.

Optionally, the delivery apparatus 202 may further incorporate an inflatable balloon. The inflatable balloon can be configured to apply a radially expanding force to radially-expandable prosthetic heart valve 210 to expand it from a radially-compressed condition to a fully-expanded condition within the native heart valve 212. In an alternative example, the prosthetic heart delivery system may be configured for use with a self-expanding prosthetic heart valve, and the inflatable balloon may be omitted.

With continued reference to FIG. 9, the delivery apparatus 202 can deliver the radially-expandable prosthetic heart valve 210 to an installation site located at a native heart valve 212. In such a procedure, the guidewire 208 may through the native heart valve 212, and the delivery apparatus 202 may travel along the guidewire 208 towards the distal end 214 of the guidewire 208, carrying the radially-expandable prosthetic heart valve 210 to the native heart valve 212. The radially-expandable prosthetic heart valve 210 may then be expanded such that its outer diameter matches the inner diameter of the native heart valve 212 may be expanded, for example by inflating a balloon such as balloon 14 from FIGS. 1-5. In alternative examples, the radially-expandable prosthetic heart valve 210 may be self-expanding.

In certain examples of a prosthetic heart valve delivery system having a multiple location pressure sensing device, the pressure sensors on the guidewire and/or delivery apparatus may be positioned near the inflow and the outflow of the valve. To achieve this, the two or more sensors may be placed, for example both on the guidewire, both on the delivery apparatus, or one on the guidewire and one on the delivery apparatus. It is to be understood that in examples having more than two sensors, any additional sensors may be placed on the guidewire, the delivery apparatus, or both.

In some examples, both sensors of the multiple location pressure sensing device may be located on the guidewire. In one example of a prosthetic heart valve delivery assembly 100 having a multiple location pressure sensing device with both sensors on the guidewire, shown in FIG. 6, a distal sensor 102 may be located towards a distal end 112 of a guidewire 110, and a proximal sensor 104 may be located on a proximal region 114 of the guidewire 110 that is closer to the user than the distal end 112 of the guidewire 110. In such a configuration, the distal sensor 102 and the proximal sensor 104 may be spaced apart from one another such that the sensors may be on opposite sides of a prosthetic heart valve 106 that has been installed in native heart valve 108 of a patient. In such examples of a prosthetic heart valve delivery assembly having a multiple location pressure sensing device, one sensor may be positioned on each side of the prosthetic heart valve 106 by, for example, leaving the guidewire in place after the prosthetic heart valve 106 has been installed. Thus, the distal sensor 102 will be positioned on one side of the prosthetic heart valve and the proximal sensor 104 will be positioned on the other, allowing for the calculation of a pressure gradient across the prosthetic heart valve 106.

It is to be appreciated that the orientation of the inlet end 116 and outlet end 118 of prosthetic heart valve 106 relative to the distal end 112 and the proximal region 114 of the guidewire 110 may depend on the specific native heart valve being replaced and the nature of the replacement operation. For example in some examples, such as the one shown in FIG. 6, an inlet end 116 of the prosthetic heart valve 106 may be installed facing towards the distal end 112 of the guidewire 110 and the outlet end 118 of the prosthetic heart valve may be installed facing towards the proximal region 114 of the guidewire 110. It is to be appreciated, however, that in other examples, the inlet end of the prosthetic heart valve may be installed facing towards the proximal region of the guidewire, and the outlet end of the prosthetic heart valve may be installed facing towards the distal end of the guidewire.

One notable advantage of examples having both sensors on the guidewire is the reduced obstruction of a heart valve annulus during measurement. A minimized obstruction in turn may reduce the error induced in the pressure measurements by the obstruction. In particular, because the guidewire has a smaller diameter than either the delivery apparatus and/or inflatable balloon which run along it, it offers the smallest possible obstruction to the heart valve annulus during measurement. Furthermore, the guidewire may be more easily manipulated within the body of the patient than other components of the delivery assembly, reducing the chance of complications associated with repositioning components of the delivery assembly.

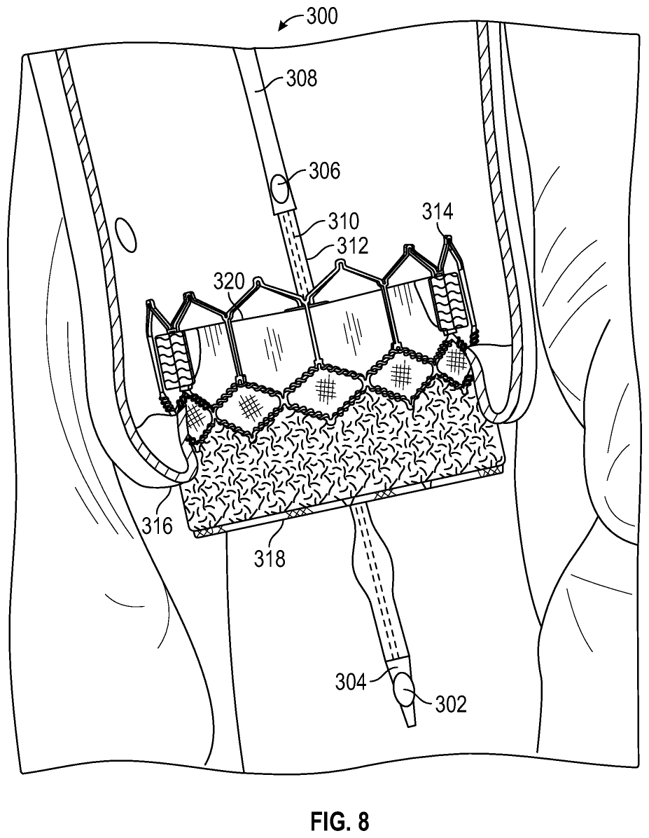

In other examples of a prosthetic heart valve delivery assembly having a multiple location pressure sensing device, both sensors may be located on components of the delivery apparatus. In one such example, shown in FIG. 8 a prosthetic heart valve delivery assembly 300 can comprise a distal sensor 302 located on a nosecone 304 of a delivery apparatus, and a proximal sensor 306 located on a nosecone shaft 308 of a delivery apparatus. The prosthetic heart valve delivery assembly 300 may further comprise guidewire 310 and a delivery sheath 312. In such examples, the nosecone 304 and the nosecone shaft 308 may be independently positionable along the length of the guidewire 310 and the delivery sheath 312, such that the distal sensor 302 and the proximal sensor 306 may be positioned on either side of a prosthetic heart valve 314 after it has been expanded within native heart valve 316.

To obtain a measurement of the pressure gradient using the multiple location pressure sensing device according to this example, the nosecone 304 may be left on the distal side of the prosthetic heart valve after installation, while the nosecone shaft 308 is drawn back to proximal side of the prosthetic heart valve as shown in FIG. 8. Measurements of pressure may then be taken on both sides of the prosthetic heart valve, and a pressure gradient across the valve may be calculated.

It is to be appreciated that the orientation of the inlet end 318 and outlet end 320 prosthetic heart valve 314 with respect to the distal sensor and the proximal sensor may depend on the valve being replaced. For example in some examples, such as the one shown in FIG. 8, the inlet end 318 of the prosthetic heart valve 314 may be installed facing towards the distal end of the guidewire 310 and the outlet end 320 of the prosthetic heart valve 314 may be installed facing towards the proximal region of the guidewire 310. In other examples, the inlet end 318 of the prosthetic heart valve 314 may be installed facing towards the proximal region of the guidewire 310, and the outlet end 320 of the prosthetic heart valve may be installed facing towards the distal end of the guidewire 310.

In yet other examples of a prosthetic heart valve delivery assembly having a multiple location pressure sensing device, one sensor may be located on the guidewire and another sensor may be located on the delivery apparatus. In one such example, shown in FIG. 7, a prosthetic heart valve delivery assembly 400 can comprise a distal sensor 402 located on a nosecone 404 of a delivery apparatus and a proximal sensor 406 located on a guidewire 408 or a delivery sheath 410. The prosthetic heart valve delivery assembly 400 may further comprise a nosecone shaft 412. In such examples, the nosecone 404 may be configured to freely move along guidewire 408 and/or delivery sheath 410, thereby allowing the distal sensor 402 and the proximal sensor 406 to be independently movable relative to one another so that they may be positioned on either side of prosthetic heart valve 414 after it has been expanded within native heart valve 416.

To obtain a measurement of the pressure gradient using the multiple location pressure sensing device according to this example, the nosecone 304 may be left on the distal side of the prosthetic heart valve after installation, while the nosecone shaft 308 is drawn back to proximal side of the prosthetic heart valve as shown in FIG. 7. Measurements of pressure may then be taken on both sides of the prosthetic heart valve, and a pressure gradient across the valve may be calculated.

To obtain a measurement of the pressure gradient across prosthetic heart valve 414, one sensor may be positioned on each side of the prosthetic heart valve 414 by, for example, leaving the distal sensor on the distal side of the prosthetic heart valve 414 and either passing the nosecone 404 through the prosthetic heart valve towards the distal end of the guidewire 408 or by leaving the nosecone 404 extended through the prosthetic heart valve 414 after the installation process is complete, as is shown in FIG. 7. Thus, the distal sensor 402 will be positioned on one side of the prosthetic heart valve and the proximal sensor 406 will be positioned on the other, allowing for the calculation of a pressure gradient across the prosthetic heart valve 414.

It is to be appreciated that the orientation of the inlet end 418 and outlet end 420 prosthetic heart valve 414 with respect to the distal sensor and the proximal sensor may depend on the valve being replaced. For example in some examples, such as the one shown in FIG. 7, the inlet end 418 of the prosthetic heart valve 414 may be installed facing towards the distal end of the guidewire 408 and the outlet end 420 of the prosthetic heart valve 414 may be installed facing towards the proximal region of the guidewire 408. In other examples, the inlet end 418 of the prosthetic heart valve 414 may be installed facing towards the proximal region of the guidewire 408, and the outlet end 420 of the prosthetic heart valve may be installed facing towards the distal end of the guidewire 408.

Returning to FIG. 9, another example of a prosthetic heart valve delivery assembly 200 having a multiple location pressure sensing device may comprise a guidewire 208 and a delivery apparatus 202 comprising a nosecone 204 and a nosecone shaft 206. The prosthetic heart valve delivery assembly 200 can further comprise a distal sensor 216 located on a distal end 214 of the guidewire 208 and a proximal sensor 218 located on the nosecone shaft 206.

In the example of the prosthetic heart valve delivery assembly having a multiple location pressure sensing device shown in FIG. 9, one sensor may be positioned on each side of the prosthetic heart valve by, for example, retracting the delivery apparatus 202 comprising the nosecone 204 and the nosecone shaft 206 towards the proximal end of the guidewire 208, while leaving the distal end of the guidewire 208 extended through the prosthetic heart valve 210 after the installation is complete. Thus, the proximal sensor 218 will be positioned on one side of the prosthetic heart valve 210 and the distal sensor 216 will be positioned on the other, allowing for the calculation of a pressure gradient across the prosthetic heart valve 210.

It is to be appreciated that the orientation of the inlet end 220 and outlet end 222 prosthetic heart valve 210 with respect to the distal sensor 216 and the proximal sensor 218 may depend on the valve being replaced. For example in some examples, such as the one shown in FIG. 9, the inlet end 220 of the prosthetic heart valve 210 may be installed facing towards the distal end of the guidewire 208 and the outlet end 222 of the prosthetic heart valve 210 may be installed facing towards the proximal region of the guidewire 208 In other examples, the inlet end 220 of the prosthetic heart valve 210 may be installed facing towards the proximal region of the guidewire 208, and the outlet end 222 of the prosthetic heart valve 210 may be installed facing towards the distal end of the guidewire 208.

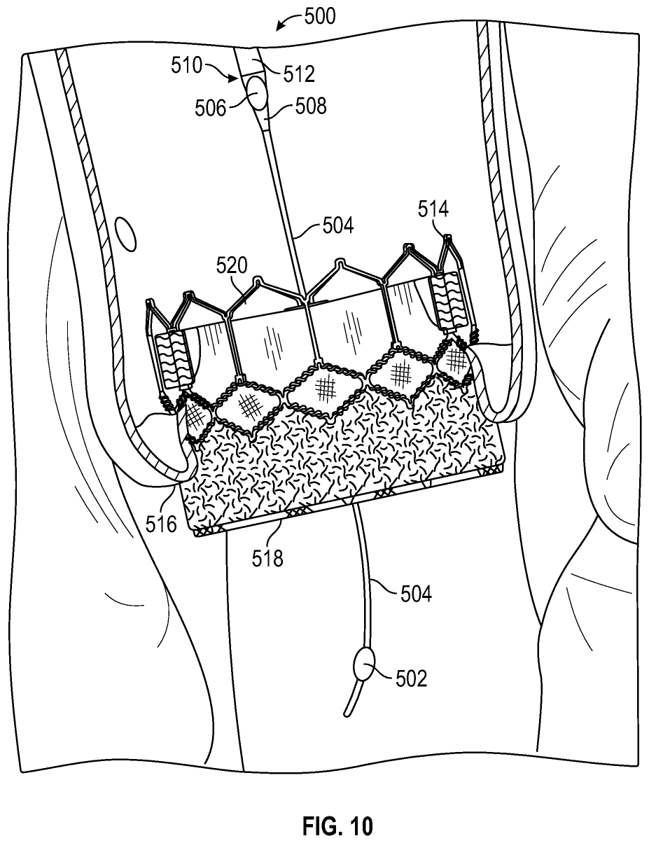

In an alternative example shown in FIG. 10, prosthetic heart valve delivery assembly 500 having a multiple location pressure sensing device may comprise distal sensor 502 positioned on the distal end of guidewire 504 and proximal sensor 506 positioned on nosecone 508 of the delivery apparatus 510. In some examples, the delivery apparatus 510 may further comprise a nosecone shaft 512. In such a configuration, the sensors may be spaced apart from one another such that the sensors may be on opposite sides of a prosthetic heart valve 514 that has been installed in a native heart valve 516 of a patient.

In the example of the prosthetic heart valve delivery assembly having a multiple location pressure sensing device shown in FIG. 10, one sensor may be positioned on each side of the prosthetic heart valve by, for example, retracting the delivery apparatus 510 comprising the nosecone 508 and the nosecone shaft 512 towards the proximal end of the guidewire 504, while leaving the distal end of the guidewire 504 extended through the prosthetic heart valve 514, after the installation is complete. Thus, the proximal sensor 506 will be positioned on one side of the prosthetic heart valve and the distal sensor 502 will be positioned on the other, allowing for the calculation of a pressure gradient across the prosthetic heart valve 514.

It is to be appreciated that the orientation of the inlet end 518 and outlet end 520 prosthetic heart valve 514 with respect to the distal sensor 502 and the proximal sensor 506 may depend on the valve being replaced. For example in some examples, such as the one shown in FIG. 9, the inlet end 518 of the prosthetic heart valve 514 may be installed facing towards the distal end of the guidewire 504 and the outlet end 520 of the prosthetic heart valve 514 may be installed facing towards the proximal region of the guidewire 208 In other examples, the inlet end 518 of the prosthetic heart valve 514 may be installed facing towards the proximal region of the guidewire 504, and the outlet end 520 of the prosthetic heart valve 514 may be installed facing towards the distal end of the guidewire 504.

One notable advantage of examples of a prosthetic heart valve delivery assembly with a multiple location pressure sensor device having at least one sensor on the delivery apparatus, that is on either the delivery sheath, the nosecone shaft, or the nosecone is that this configuration enables independent positioning of the sensors. As will be discussed in greater detail below, the desired distance of the distal and proximal sensors from the inlet and outlet ends of the prosthetic heart valve at the time of pressure gradient measurement may vary from patient to patient. If the sensors are independently movable relative to one another, a physician installing the prosthetic heart valve may be able to adjust the measurement position as needed to obtain the best measurement possible.

In all such examples of a multiple location pressure sensing device having a proximal sensor and a distal sensor, the pressure gradient across a prosthetic heart valve may be calculated from the difference in simultaneous pressure measurements taken by the proximal sensor and the distal sensor. It is to be appreciated that, dependent on which valve is being replaced, as discussed above, the proximal sensor may measure the inlet pressure and the distal sensor may measure the outlet pressure, or the distal sensor may measure inlet pressure and the proximal sensor may measure the outlet pressure. While pressure gradient may, for example, be measured by subtracting the outlet pressure from the inlet pressure, it is to be appreciated that under certain conditions, a physician may also wish to measure a pressure gradient in the other direction by subtracting the inlet pressure from the outlet pressure.

In most examples disclosed herein, pressure sensors are added to one or more components that already enter the heart in the course of deploying the prosthetic heart valve. Advantageously, in such examples no new additional components must be introduced into the heart to take the pressure measurements, and no further complexity is added to the operation of installing a prosthetic heart valve in a patient.

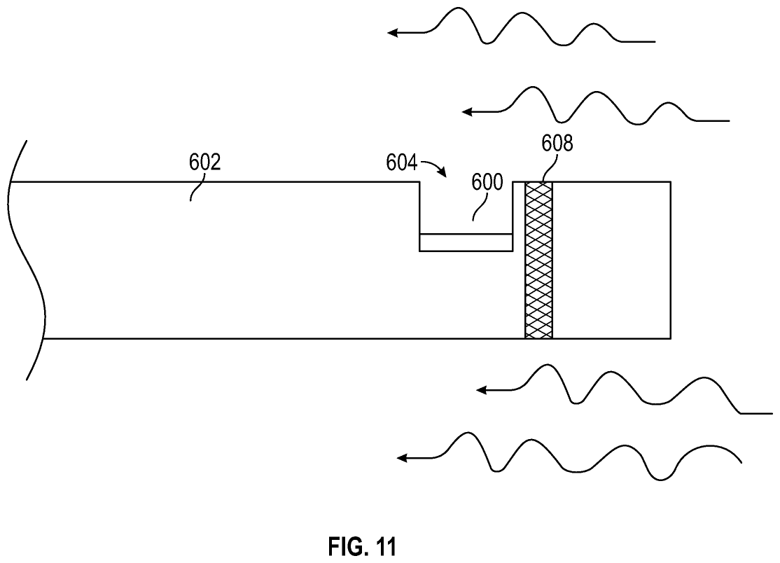

The sensors of the multiple location pressure sensing device examples disclosed herein may be recessed into any of the abovementioned components of the prosthetic heart delivery assembly. As best shown in FIG. 11, a pressure sensor 600 may be recessed into a guidewire 602 and exposed to the blood flow in a patient's heart by means of a sensor window 604 formed in the guidewire 602. While FIG. 11 shows a sensor inset into a guidewire, it is to be understood that the sensor may also be inset in another structural component of prosthetic heart valve delivery assembly, such as a nosecone, a delivery sheath, or a nosecone shaft.

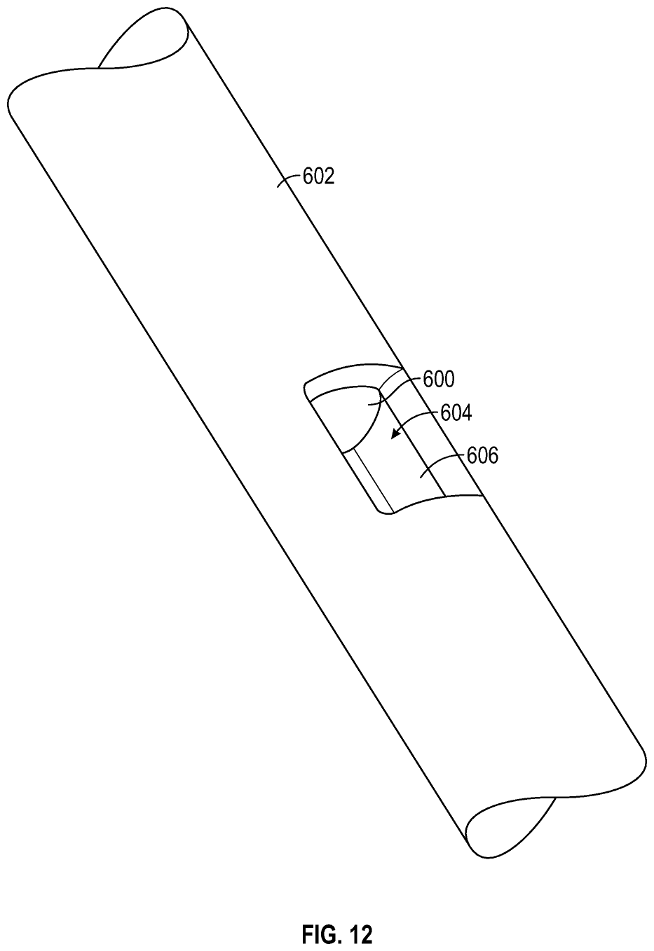

As further illustrated in FIG. 12, the sensor window 604 may be formed in the exterior surface of the guidewire 602 and may expose a recess 606 containing a pressure sensor 600. The depth of the recess may, in some examples, be half or less than half of the total thickness of the guidewire, such as one third or one quarter the total thickness of the guidewire.

While FIGS. 11 and 12 depict an example in which the sensor is recessed in a component of the prosthetic heart valve delivery assembly, such as the guidewire, it is to be understood that in other examples, the two or more pressure sensors may be disposed on the surface of a component of the prosthetic heart valve delivery assembly, such as the surface of the guidewire, the surface of the delivery sheath, the surface of the nosecone, or the surface of the nosecone shaft.

In some examples, the inlet sensor and outlet sensor may be positioned at a chosen distance away from the inlet and outlet ends of the installed prosthetic heart valve, respectively. In many cases, the ideal distance between the sensors and the ends of the prosthetic heart valves may be clinically-determined, and this may vary from patient to patient, but generally will be 7 cm or less (or within 1-5 cm in particular examples). For example, in some instances, the inlet sensor may be positioned within 7 cm of the inlet end of the prosthetic heart valve, such as 1 cm, 2 cm, 3 cm, 4 cm, 5 cm, 6 cm, or 7 cm away from the inlet end of the prosthetic heart valve. In some examples, the outlet sensor may be positioned within 7 cm of the outlet end of the prosthetic heart valve, such as 1 cm, 2 cm, 3 cm, 4 cm, 5 cm, 6 cm, or 7 cm from the outlet end of the prosthetic heart valve. It is to be understood that the examples listed above can be used in any suitable combination.

In certain specific examples previously mentioned, the sensors may be configured to be on components of the delivery assembly that are independently movable relative to one another, such as one sensor on the guidewire and one sensor on the delivery sheath, or one sensor on the guidewire and one sensor on a different component of the delivery apparatus, or one sensor on the nosecone shaft of the delivery apparatus and one sensor on the nosecone of the delivery apparatus. It is to be appreciated that this functionality may be possible with any example having a sensor configuration in which the sensors may be moved. This configuration may offer certain advantages, such as offering a physician the ability to change the relative position of each sensor with respect to the prosthetic heart valve, or to change the distance separating the inlet sensor and the outlet sensor according to the needs of the patient on an operation-by-operation basis.

In some alternative examples, more than two pressure sensors disposed in an array can be employed such that data is gathered from which to generate a pressure map that may optionally be displayed on a display screen. Depending on the number and placement of the sensors, one, two, and/or three-dimensional representations of pressure data can be made. Sensors may be arranged in order to simultaneously measure pressure data in more than two locations in the heart.

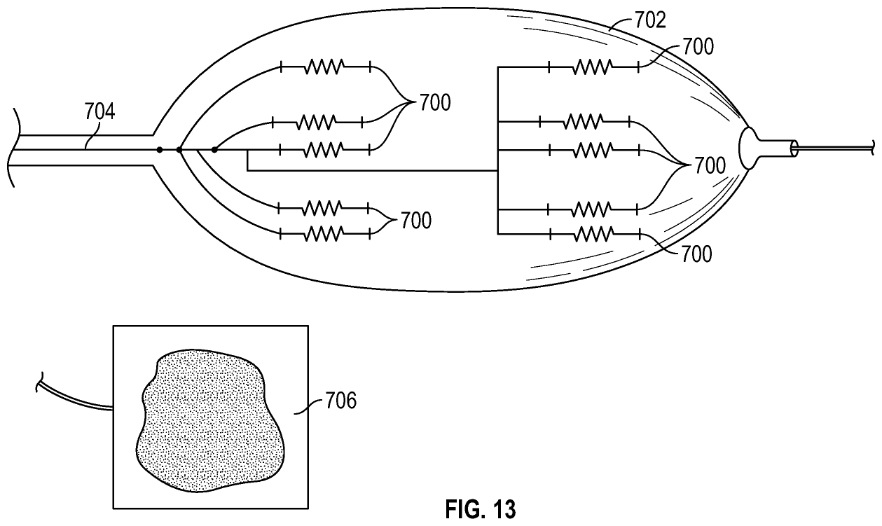

One alternative example is illustrated in FIG. 13. In this example, pressure sensors 700 are located at various positions on a balloon 702 for expanding a prosthetic heart valve into place in the heart. Some sensors are located on a proximal portion of the balloon, while other sensors are located on a distal portion of the balloon. In FIG. 11, the valvular structure of the prosthetic heart valve has been omitted for clarity.

The array of pressure sensors 700 can together send pressure data back to a processing unit via a wire lead 704. From the pressure sensor data, a pressure map or other graphic may be generated and displayed on a screen of a display unit 706. The sensors may sense different pressures at different locations on the surface of the balloon. Although the prosthetic heart valve is shown in the drawings as being balloon-expandable, it is to be understood that self-expandable prosthetic heart valves may also be used.

In one example of a delivery assembly having a multiple location pressure measurement system, the multiple location pressure measurement system may include a control module that is located outside of the patient, either on the table or on a stand or rack. The control module can be connected to the proximal end of the guidewire and/or the delivery assembly. The control module may include a zeroing feature to allow the physician to set baseline atmospheric pressure. The control module may, for example, display and record the gradient or difference in pressure between the two or more sensors. The control module could also provide any input power to the sensor and interface with other cath lab hemodynamic equipment.

In certain examples of the multiple location pressure sensing devices disclosed herein, the location of the sensors may be indicated by a radiopaque marker located on or near the sensor and/or sensor window. For example, as illustrated in FIG. 11, a guidewire 602 may comprise a radiopaque marker 608 disposed near a sensor window 604 exposing a pressure sensor 600 to blood flow. While FIG. 11 shows a radiopaque marker disposed on the guidewire of a prosthetic heart valve delivery assembly having a pressure sensor located on the guidewire, it is to be understood that, in examples having the pressure sensors located on other components such as the nosecone, nosecone shaft, or delivery sheath, the radiopaque marker may be located on such components as well.

Advantageously, the inclusion of a radiopaque marker may allow a physician to identify the location of the guidewire, delivery sheath, and/or sensors during the installation process, thereby facilitating the correct siting of the pressure sensors during the installation process and measurement of the pressure gradient thereafter.

A multiple location pressure sensing device may further comprise a data display or readout. In examples having a data display, the data display may generally be positioned outside the body of the patient, and configured to receive data transmitted from the two or more pressure sensors and display it in a format readable by, for example, a physician conducting a prosthetic heart valve replacement procedure.

In some examples, the multiple location pressure sensing device is configured to wirelessly transmit pressure measurements. In such examples, the prosthetic heart valve delivery assembly may further comprise a wireless transmitter and a wireless receiver. The wireless transmitter may be located, for example, on the guidewire, on the nosecone or the nosecone shaft of the delivery apparatus, or on the delivery sheath. Generally, the wireless receiver is positioned outside the body of the patient, and may optionally be in communication with a display device that allows for

Examples of the disclosed technology may have additional or alternative features. For instance, data from the pressure sensors may be communicated via Bluetooth. The Bluetooth chip may be located, for example, near the pressure sensors. In another example, an electrical lead may extend from the pressure sensor to a Bluetooth chip on a handle of the delivery assembly, from which a signal is transmitted to a Bluetooth receiver.

With wireless data transfer capabilities, these flexible sensors offer a noninvasive way to monitor the functioning of implanted medical devices. Such sensors may be adapted to monitor pressure differentials, as discussed herein.

Considering particularly examples in which a guidewire includes one or more pressure sensors, one purpose of a guidewire is to provide a pathway for another device to track over it. Many delivery assemblies have a small guidewire lumen running through the center of the device so it may ride over a guidewire, to navigate the patient's vasculature into a location in the heart, such as the aortic valve. Consequently, the guidewire may be detachable from the control unit so as to facilitate aspects of the procedure.

Also disclosed herein are methods by which a prosthetic heart valve delivery assembly having a multiple location pressure sensor may be employed to determine the pressure gradient across a prosthetic heart valve. Generally, a pressure gradient determination procedure consists of positioning the pressure sensors in the desired location, exposing the sensors to the blood flow within the heart, measuring pressure at both ends of the prosthetic heart valve, and calculating the pressure gradient across the prosthetic heart valve. Optionally, the calculated gradient may be used to adjust the installation of the prosthetic heart valve.

In one example of a pressure gradient determination procedure, following the installation of the prosthetic heart valve, the inlet sensor is positioned near the inlet end of the installed prosthetic heart valve, and the outlet sensor is positioned near the outlet end of the installed prosthetic heart valve. In such an example, once the inlet sensor and outlet sensor have been located near the inlet and outlet ends of the prosthetic heart valve, pressure measurements are taken by exposing the inlet sensor and the outlet sensor to the blood stream.

In one particular example, continuous measurements are taken of the pressure on each side of the installed prosthetic heart valve. The measurements may be used to identify the time at which the heart is at ventricular systole, which will correspond generally to the maximum pressure gradient across the installed prosthetic heart valve. The difference in pressure measured by the inlet sensor and outlet sensor may then be calculated to determine the pressure gradient across the heart valve. In another example, continuous measurements may be used to determine when the heart is at ventricular diastole, and a pressure gradient may be measured across the prosthetic heart valve at the point of ventricular diastole. In an alternative method example, single measurements may be taken instead of continuous measurements. In such an alternative method example, the pressure gradient may be calculated from the single measurement, without the step of identifying the point of either ventricular systole or ventricular diastole.

As previously discussed, pressure data may be transmitted from the pressure sensors by means of a cable, such as a fiberoptic cable or an electrical cable that is incorporated into the guidewire, and runs along the length of the guidewire from the sensors to a monitor outside the body of the patient. In other examples, however, the data may be communicated wirelessly, from a transmitter included in the delivery assembly to a wireless receiver located outside the patient.

Once the pressure gradient across the prosthetic heart valve is known, a physician may compare the measured gradient against a pressure gradient limit. This pressure gradient limit may depend on factors, such as patient health, the specific native heart valve being replaced, and other relevant medical factors, which in some instances can be less than 14 mm Hg, or less than 8 mm Hg. If the measured pressure gradient is observed to be outside the acceptable limits for the procedure, the valve may be re-expanded. In the case of a mechanically-expanded prosthetic heart valve, an expansion balloon may be repositioned inside the prosthetic heart valve and expanded to a new diameter greater than the present diameter of the prosthetic heart valve. It is to be appreciated that, in the case of a self-expanding prosthetic heart valve, other mechanisms may be used to re-expand the prosthetic heart valve. Once the prosthetic heart valve has been re-expanded, the pressure gradient can be re-measured by the same methods discussed above, until the measured pressure gradient across the prosthetic heart valve is below the maximum allowable limit.

The prosthetic heart valve delivery assemblies with multiple location pressure sensing systems and associated methods of use disclosed herein may be used with various examples of prosthetic heart valves. Prosthetic heart valves for use with the presently disclosed prosthetic heart valve delivery assemblies with multiple location pressure sensing devices can have a frame assembly comprising at least one radially compressible and expandable frame and a valvular structure supported within the frame assembly. Additionally, the prosthetic heart valves may have a plurality of anchoring structures for securing the prosthetic heart valve to native tissue of a patient. In some examples, the frame assembly can comprise an inner frame and an outer frame.

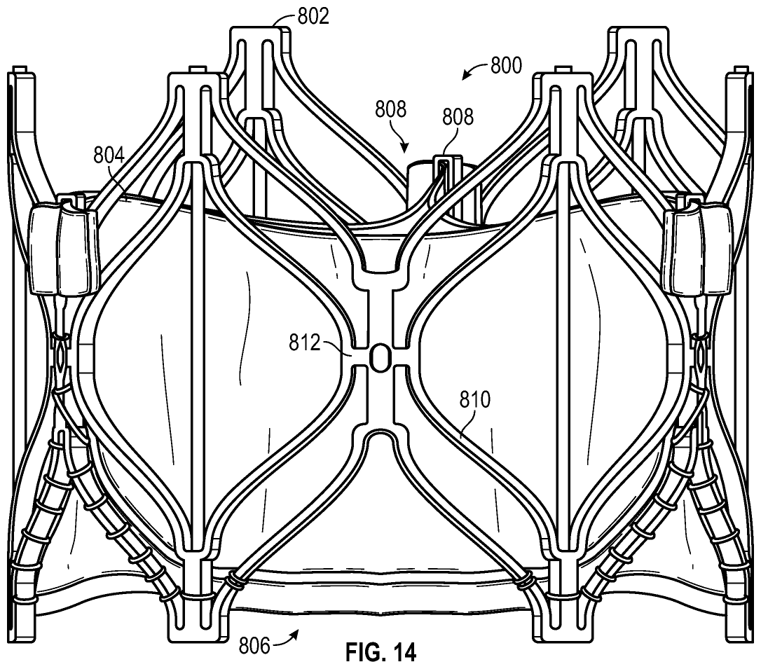

For example, FIG. 16 depicts one example of a prosthetic heart valve 800 that can be used with the multiple location pressure sensing devices disclosed herein. The prosthetic heart valve 800 comprises a frame assembly 802 and a valvular structure 804 supported by the frame assembly 802. The frame assembly 802 defines an inflow end 806 and an outflow end 808 of the prosthetic heart valve 800. As shown in FIG. 14, the frame assembly 802 in the illustrated example comprises a plurality of interconnected struts 810 arranged in a lattice or diamond pattern. In some prosthetic heart valve examples suitable for use with the multiple location pressure sensing devices disclosed herein, as shown in FIG. 14, the interconnected struts 810 of the frame assembly 802 can be connected at stationary junctions 812. The frame assembly 802 can have a generally cylindrical shape such that it has a substantially constant diameter from an upper end (inlet end) to a lower end (outlet end) of the frame assembly 802. However, it is to be understood that in alternative examples, the diameter of the frame assembly 802 can vary along its length. Although frame assembly 802 is described as generally having a cylindrical shape, it is to be understood that all or a portion of the frame assembly 802 can have a non-circular cross-section such as, but not limited to, a D-shape.

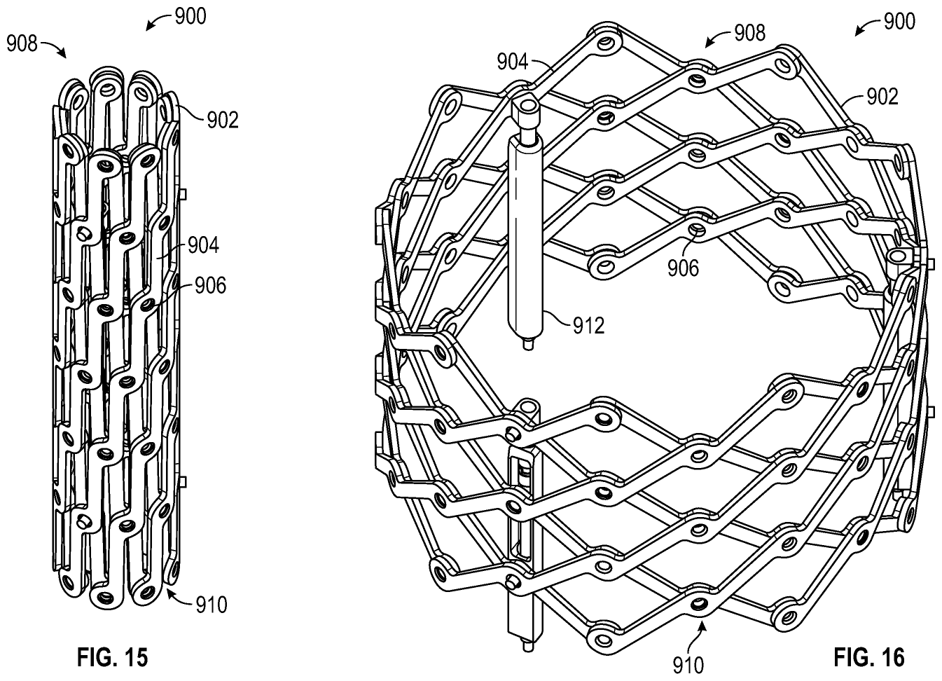

In other prosthetic heart valve examples suitable for use with the prosthetic heart valve delivery assemblies with multiple location pressure sensing devices disclosed herein, best illustrated in FIGS. 15 and 16, the frame assembly can further comprise joints or pivots between the struts. For example, as illustrated in FIG. 15, a prosthetic heart valve 900 may comprise a frame assembly 902 having a plurality of struts 904 arranged in a lattice or diamond pattern and a valvular structure supported by the frame. The plurality of struts 904 can connect at a plurality of pivot joints 906, configured to allow connected struts 904 to move axially and rotationally relative to one another. In this way, the frame assembly 902 of prosthetic heart valve 900 may be configured to radially expand from a fully-collapsed configuration shown in FIG. 15 to a fully-expanded configuration as shown in FIG. 16 as axial compressive force is applied to the inlet end 908 and the outlet end 910 of the frame, for example as applied by one or more actuators 912 (which can also be referred to as “lockers” and/or “expansion mechanisms”), or as a radially expansive force is applied to the frame.

In prosthetic heart valve examples having a frame comprising struts 904 and pivot joints 906, the frame may further comprise locking elements included in the one or more actuators 912, configured to arrest the motion of the pivot joints 906 of the frame assembly 902 when the prosthetic heart valve is in a fully expanded configuration.

### ADDITIONAL EXAMPLES OF THE DISCLOSED TECHNOLOGY

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. A delivery assembly for a prosthetic heart valve, comprising a delivery apparatus, a guidewire, a first pressure sensor, and a second pressure sensor. The guidewire extends through the delivery apparatus. The first pressure sensor is configured to be positioned near an inlet end of a prosthetic heart valve, and the second pressure sensor is configured to be positioned near an outlet end of the prosthetic heart valve. The first pressure sensor and second pressure sensor are positioned on the delivery apparatus or the guidewire and are configured to measure a pressure gradient across the prosthetic heart valve.
Example 2. The delivery assembly of any example herein, particularly example 1, wherein the prosthetic heart valve is positioned between a right atrium and a right ventricle of a patient's heart.
Example 3. The delivery assembly of any example herein, particularly example 1, wherein the prosthetic heart valve is positioned between a left atrium and a left ventricle of a patient's heart.
Example 4. The delivery assembly of any example herein, particularly example 1, wherein the prosthetic heart valve is positioned between a left ventricle and an aorta of a patient's heart.
Example 5. The delivery assembly of any example herein, particularly example 1, wherein the prosthetic heart valve is positioned between a right ventricle and a pulmonary artery of a patient.
Example 6. The delivery assembly of any example herein, particularly any one of examples 1-5, wherein the first pressure sensor and the second pressure sensor are positioned on the guidewire.
Example 7. The delivery assembly of any example herein, particularly example 6, wherein at least one of the first pressure sensor and the second pressure sensor are inset in the guidewire.
Example 8. The delivery assembly any example herein, particularly any one of examples 1-7, wherein the first pressure sensor is positioned on the guidewire, and wherein the second pressure sensor is positioned on the delivery apparatus.
Example 9. The delivery assembly of any example herein, particularly any one of examples 1-8, wherein the first pressure sensor is positioned on the delivery apparatus, and wherein the second pressure sensor is positioned on the guidewire.
Example 10. The delivery assembly of example 1, wherein the first pressure sensor and the second pressure sensor are positioned on the delivery apparatus.
Example 11. The delivery assembly of any example herein, particularly example 1, wherein the delivery apparatus further comprises an inflatable balloon.
Example 12. The delivery assembly of any example herein, particularly example 1, wherein the delivery apparatus further comprises an outer sheath, a nosecone, and a nosecone shaft.
Example 13. The delivery assembly of any example herein, particularly example 12, wherein the first pressure sensor is positioned on the guidewire, and wherein the second pressure sensor is positioned on the nosecone shaft.
Example 14. The delivery assembly of any example herein, particularly example 12, wherein the first pressure sensor is positioned on the nosecone shaft and the second pressure sensor is positioned on the guidewire.
Example 15. The delivery assembly of any example herein, particularly example 12, wherein the first pressure sensor is positioned on the guidewire, and wherein the second pressure sensor is positioned on the nosecone.
Example 16. The delivery assembly of any example herein, particularly example 12, wherein the first pressure sensor is positioned on the nosecone, and wherein the second pressure sensor is positioned on the guidewire.
Example 17. The delivery assembly of any example herein, particularly example 12, wherein the first pressure sensor is positioned on the nosecone, and wherein the second pressure sensor is positioned on the nosecone shaft.
Example 18. The delivery assembly of any example herein, particularly example 12, wherein the first pressure sensor is positioned on the nosecone shaft, and wherein the second pressure sensor is positioned on the guidewire.
Example 19. The delivery assembly of any example herein, particularly any one of examples 1-18, wherein the delivery assembly further comprises a fiberoptic cable for transmitting data from the first pressure sensor and the second pressure sensor.
Example 20. The delivery assembly of any example herein, particularly any one of examples 1-19, wherein the delivery assembly further comprises an electrical wire for transmitting data from the first pressure sensor and the second pressure sensor.
Example 21. The delivery assembly of any example herein, particularly any one of examples 1-20, wherein the delivery assembly further comprises a wireless device for transmitting data from the first pressure sensor and the second pressure sensor and a wireless receiver to receive the transmitted data.
Example 22. The delivery assembly of any example herein, particularly any one of examples 1-21, wherein the delivery assembly comprises more than two pressure sensors.
Example 23. The delivery assembly of any example herein, particularly any one of examples 1-22, wherein the pressure sensors are optical pressure sensors.
Example 24. The delivery assembly of any example herein, particularly any one of examples 1-23, wherein the pressure sensors are piezoelectric pressure sensors.
Example 25. The delivery assembly of any example herein, particularly any one of examples 1-24, wherein the pressure sensors are independently movable relative to one another.
Example 26. The delivery assembly of any example herein, particularly any one of examples 1-25, wherein the delivery assembly further comprises one or more radiopaque markers.
Example 27. The delivery assembly of any example herein, particularly any one of examples 1-26, wherein the first pressure sensor is configured to take measurements within a range of 0-7 cm or 1-5 cm from the inlet end of the prosthetic heart valve.
Example 28. The delivery assembly of any example herein, particularly any one of examples 1-27, wherein the second pressure sensor is configured to take measurements within a range of 0-7 cm or 1-5 cm from the outlet end of the prosthetic heart valve.
Example 29. The delivery assembly of any example herein, particularly any one of examples 1-28, wherein the delivery assembly further comprises a display apparatus configured to display the measurements taken by at least the first pressure sensor and the second pressure sensor.
Example 30. A method of measuring a pressure gradient across a prosthetic heart valve, comprising placing a first pressure sensor at a first sensor location near an inlet end of a prosthetic heart valve installed in a heart of a patient, placing a second pressure sensor at a second sensor location near an outlet end of the prosthetic heart valve installed in the patient, simultaneously measuring a first pressure at the first sensor location and a second pressure at the second sensor location, and calculating the pressure gradient across the prosthetic heart valve from the first pressure measured at the first sensor location and the second pressure measured at the second sensor location.
Example 31. The method of any example herein, particularly example 30, wherein the first sensor location is in a left ventricle of the patient, and wherein the second sensor location is in an aorta of the patient.
Example 32. The method of any example herein, particularly example 30, wherein the first sensor location is in a left atrium of the patient, and wherein the second sensor location is in a left ventricle of the patient.
Example 33. The method of any example herein, particularly example 30, wherein the first sensor location is in a right atrium of the patient, and wherein the second sensor location is in a right ventricle of the patient.
Example 34. The method of any example herein, particularly example 30, wherein the first sensor location is in the right ventricle of the patient, and wherein the second sensor location is in the pulmonary artery of the patient.
Example 35. The method of any example herein, particularly any one of examples 30-34, wherein the first pressure sensor and the second pressure sensor are placed by positioning a guidewire.
Example 36. The method of any example herein, particularly any one of examples 30-34, wherein the first pressure sensor is placed by positioning a guidewire, and wherein the second pressure sensor is placed by positioning a delivery apparatus.
Example 37. The method of any example herein, particularly any one of examples 30-34, wherein the first pressure sensor is placed by positioning a delivery apparatus, and wherein the second pressure sensor is placed by positioning a guidewire.
Example 38. The method of any example herein, particularly any one of examples 30-34, wherein the first pressure sensor and the second pressure sensor are placed by positioning a delivery apparatus.
Example 39. The method of any example herein, particularly any one of examples 30-34, wherein the first pressure sensor and the second pressure sensor are placed by positioning a delivery apparatus.
Example 40. The method of any example herein, particularly any one of examples 30-34, wherein one of the first pressure sensor and the second pressure sensor is placed by positioning a delivery apparatus, and wherein the other pressure sensor is placed by positioning a guidewire.
Example 41. The method of any example herein, particularly any one of examples 30-34, wherein one of the first pressure sensor and the second pressure sensor is placed by positioning a delivery apparatus, and wherein the other pressure sensors are placed by positioning a delivery apparatus having a nosecone and a nosecone shaft.
Example 42. The method of any example herein, particularly example 41, wherein the first pressure sensor is placed by positioning the nosecone of the delivery apparatus, and wherein the second pressure sensor is placed by positioning the nosecone shaft of the delivery apparatus.
Example 43. The method of any example herein, particularly any one of examples 30-42, further comprising measuring the first pressure at the first sensor location and the second pressure at the second sensor location as a function time.
Example 44. The method of any example herein, particularly example 43, wherein the first pressure at the first sensor location and the second pressure at the second sensor location are used to identify when the heart of the patient is in ventricular systole.
Example 45. The method of any example herein, particularly example 44, wherein the pressure gradient is calculated using the first pressure and the second pressure measured while the heart of the patient is in ventricular systole.
Example 46. The method of any example herein, particularly example 43, wherein the first pressure at the first sensor location and the second pressure at the second sensor location are used to identify when the heart of the patient is in ventricular diastole.
Example 47. The method of any example herein, particularly example 46, wherein the pressure gradient is calculated using the first pressure and the second pressure measured while the heart of the patient is in ventricular diastole.
Example 48. The method of any example herein, particularly any one of examples 30-47, wherein the first pressure sensor is positioned within a range of 0-7 cm from the inlet end of the prosthetic heart valve.
Example 49. The method of any example herein, particularly any one of examples 30-47, wherein the second pressure sensor is positioned within a range of 0-7 cm from the outlet end of the prosthetic heart valve.
Example 50. The method of any example herein, particularly any one of examples 30-49, further comprising a transmission of pressure data from the first pressure sensor and the second pressure sensor to a display outside the body of the patient.
Example 51. The method of any example herein, particularly example 50, wherein the transmission of pressure data occurs via fiberoptic cable.
Example 52. The method of any example herein, particularly example 50, wherein the transmission of pressure data occurs via electrical wire.
Example 53. The method of any example herein, particularly example 50, wherein the transmission of pressure data occurs via wireless device.
Example 54. The method of any example herein, particularly any one of examples 30-53, wherein the first pressure at the first sensor location and the second pressure at the second sensor location are measured by optical pressure sensors.
Example 55. The method of any example herein, particularly any one of examples 30-53, wherein the first pressure at the first sensor location and the second pressure at the second sensor location are measured by piezoelectric pressure sensors.
Example 56. The method of any example herein, particularly any one of examples 30-55, wherein the first pressure sensor and the second pressure sensor are independently movable relative to one another.
Example 57. The method of any example herein, particularly any one of examples 30-56, further comprising the step of displaying the pressure data and the pressure gradient on a display.
Example 58. The method of any example herein, particularly any one of examples 30-57, wherein the position of the first pressure sensor and the position of the second pressure sensor are measured by fluoroscopy, using a radiopaque marker.
Example 59. A method of measuring a pressure gradient across a prosthetic heart valve, comprising deploying an assembly having a delivery apparatus, a guidewire, a radially expandable prosthetic heart valve, and at least two pressure sensors into a heart of a patient, expanding the prosthetic heart valve into a native heart valve of the patient, positioning a first pressure sensor at a first location in front of an inlet of the prosthetic heart valve in a direction of flow, positioning a second pressure sensor at a second location after an outlet of the prosthetic heart valve in the direction of flow, simultaneously measuring a first pressure at the first location of the first pressure sensor and a second pressure at the second location of the second pressure sensor, and calculating the pressure gradient across the prosthetic heart valve.
Example 60. The method of any example herein, particularly example 59, wherein the delivery apparatus further comprises a delivery apparatus having a nosecone and a nosecone shaft.
Example 61. The method of any example herein, particularly example 59, wherein the first pressure sensor is positioned in a left ventricle of the patient, and wherein the second pressure sensor is positioned in an aorta of the patient.
Example 62. The method of any example herein, particularly example 59, wherein the first pressure sensor is positioned in a left atrium of the patient, and wherein the second pressure sensor is positioned in a left ventricle of the patient.
Example 63. The method of any example herein, particularly example 59, wherein the first pressure sensor is positioned in a right atrium of the patient, and wherein the second pressure sensor is positioned in a right ventricle of the patient.
Example 64. The method of any example herein, particularly example 59, wherein the first pressure sensor is positioned in a right ventricle of the patient, and wherein the second pressure sensor is positioned in a pulmonary artery of the patient.
Example 65. The method of any example herein, particularly any one of examples 59-64, wherein the first pressure sensor and the second pressure sensor are placed by positioning a guidewire.
Example 66. The method of any example herein, particularly any one of examples 59-64, wherein the first pressure sensor is placed by positioning a guidewire, and wherein the second pressure sensor is placed by positioning a delivery apparatus.
Example 67. The method of any example herein, particularly any one of examples 59-64, wherein the first pressure sensor is placed by positioning a delivery apparatus, and wherein the second pressure sensor is placed by positioning a guidewire.
Example 68. The method of any example herein, particularly any one of examples 59-64, wherein the first pressure sensor and the second pressure sensor are placed by positioning a delivery apparatus.
Example 69. The method of any example herein, particularly any one of examples 59-64, wherein both of the pressure sensors are placed by positioning a delivery apparatus.
Example 70. The method of any example herein, particularly any one of examples 60-64, wherein one of the pressure sensors is placed by positioning a delivery apparatus, and wherein the other pressure sensor is placed by positioning a guidewire.
Example 71. The method of any example herein, particularly any one of examples 60-64, wherein one of the pressure sensors is placed by positioning a delivery apparatus, and wherein the other pressure sensors are placed by positioning a delivery apparatus.
Example 72. The method of any example herein, particularly any one of examples 60-64, wherein the first pressure sensor is placed by positioning a nosecone of the delivery apparatus, and wherein the second pressure sensor is placed by positioning a nosecone shaft of the delivery apparatus.
Example 73. The method of any example herein, particularly any one of examples 59-72, further comprising measuring the first pressure at the first location and the second pressure at the second location as a function time.
Example 74. The method of any example herein, particularly example 73, wherein the first pressure at the first location and the second pressure at the second location are used to identify when the heart of the patient is in ventricular systole.
Example 75. The method of any example herein, particularly example 74, wherein the pressure gradient is calculated using the first pressure at the first pressure sensor while the heart of the patient is in ventricular systole and the second pressure at the second pressure sensor while the heart of the patient is in ventricular systole.
Example 76. The method of any example herein, particularly example 73, wherein the first pressure at the first location and the second pressure at the second location are used to identify when the heart of the patient is in ventricular diastole.
Example 77. The method of any example herein, particularly example 76, wherein the pressure gradient is calculated using the first pressure at the first pressure sensor while the heart of the patient is in ventricular diastole and the second pressure at the second pressure sensor when the heart of the patient is in ventricular diastole.
Example 78. The method of any example herein, particularly any one of examples 59-77, wherein the first pressure sensor is positioned within a range of 0-7 cm from the inlet of the prosthetic heart valve.
Example 79. The method of any example herein, particularly any one of examples 59-77 wherein the second pressure sensor is positioned within a range of 0-7 cm from the outlet of the prosthetic heart valve.
Example 80. The method of any example herein, particularly any one of examples 59-79, further comprising a transmission of pressure data from the first pressure sensor and the second pressure sensor to a display outside the body of the patient.
Example 81. The method of any example herein, particularly example 80, wherein the transmission of pressure data occurs via fiberoptic cable.
Example 82. The method of any example herein, particularly example 80, wherein the transmission of pressure data occurs via electrical wire.
Example 83. The method of any example herein, particularly example 80, wherein the transmission of pressure data occurs via wireless device.
Example 84. The method of any example herein, particularly any one of examples 59-83, wherein the first pressure at the first location and the second pressure at the second location are measured by optical pressure sensors.
Example 85. The method of any example herein, particularly any one of examples 59-84, wherein the first pressure at the first location and the second pressure at the second location are measured by piezoelectric pressure sensors.
Example 86. The method of any example herein, particularly any one of examples 59-85, wherein the first pressure sensor and the second pressure sensor are independently movable relative to one another.
Example 87. The method of any example herein, particularly any one of examples 59-86, further comprising the step of displaying the pressure data and the pressure gradient on a display.
Example 88. The method of any example herein, particularly any one of examples 59-87, wherein the positions of the first pressure sensor and the second pressure sensor are measured by fluoroscopy, using a radiopaque marker.
Example 89. The method of any example herein, particularly any one of examples 59-88, further comprising a step of comparing the pressure gradient across the prosthetic heart valve against a maximum allowable pressure gradient value and verifying that the pressure gradient is less than or equal to the maximum allowable pressure gradient.
Example 90. The method of any example herein, particularly example 89, further comprising a step of, if the pressure gradient is greater than the maximum allowable pressure gradient, expanding the prosthetic heart valve an additional amount, until the pressure gradient is less than or equal to the maximum allowable pressure gradient.
Example 91. A delivery assembly for a prosthetic heart valve, comprising a delivery apparatus, a first pressure sensor, and a second pressure sensor. The first pressure sensor is configured to be positioned near an inlet end of a prosthetic heart valve, and a second pressure sensor configured to be positioned near an outlet end of the prosthetic heart valve. The first pressure sensor and second pressure sensor are positioned on the delivery apparatus and are configured to measure a pressure gradient across the prosthetic heart valve.
Example 92. The delivery assembly of any example herein, particularly example 91, wherein the delivery apparatus further comprises an inflatable balloon.
Example 93. The delivery assembly of any example herein, particularly any one of examples 91-92, wherein the delivery apparatus further comprises a nosecone, a delivery sheath and a nosecone shaft.
Example 94. The delivery assembly of any example herein, particularly example 93, wherein the first pressure sensor is positioned on the delivery sheath and the second pressure sensor is positioned on the nosecone.
Example 95. The delivery assembly of any example herein, particularly example 93, wherein the first pressure sensor is positioned on the delivery sheath and the second pressure sensor is positioned on the nosecone shaft.
Example 96. The delivery assembly of any example herein, particularly example 93, wherein the first pressure sensor is positioned on the nosecone and the second pressure sensor is positioned on the delivery sheath.
Example 97. The delivery assembly of any example herein, particularly example 95, wherein the first pressure sensor is positioned on the nosecone shaft and the second pressure sensor is positioned on the delivery sheath.
Example 98. The delivery assembly of any example herein, particularly any one of examples 91-97, wherein the first pressure sensor and the second pressure sensor are independently movable relative to each other.
Example 99. The delivery assembly of any example herein, particularly any one of examples 91-98, wherein the delivery assembly further comprises one or more radiopaque markers.
Example 100. The delivery assembly of any example herein, particularly any one of examples 91-99, wherein the delivery assembly further comprises a fiberoptic cable for transmitting data from the first pressure sensor and the second pressure sensor.
Example 101. The delivery assembly of any example herein, particularly any one of examples 91-100, wherein the first pressure sensor is configured to take measurements within a range of 0-7 cm or 1-5 cm from the inlet end of the prosthetic heart valve.
Example 102. The delivery assembly of any example herein, particularly any one of examples 91-101, wherein the second pressure sensor is configured to take measurements within a range of 0-7 cm or 1-5 cm from the outlet end of the prosthetic heart valve.
Example 103. A delivery assembly for a prosthetic heart valve, comprising a guidewire, a first pressure sensor, and a second pressure sensor. The first pressure sensor is configured to be positioned near an inlet end of a prosthetic heart valve, and the second pressure sensor is configured to be positioned near an outlet end of the prosthetic heart valve. The first pressure sensor and second pressure sensor are positioned on the guidewire and are configured to measure a pressure gradient across the prosthetic heart valve.
Example 104. The delivery assembly of any example herein, particularly example 103, wherein the first pressure sensor and the second pressure sensor are independently movable relative to each other.
Example 105. The delivery assembly of any example herein, particularly any one of examples 103-104, wherein the delivery assembly further comprises one or more radiopaque markers.
Example 106. The delivery assembly of any example herein, particularly any one of examples 103-105, wherein the delivery assembly further comprises a fiberoptic cable for transmitting data from the first pressure sensor and the second pressure sensor.
Example 107. The delivery assembly of any example herein, particularly any one of examples 103-106, wherein the first pressure sensor is configured to take measurements within a range of 0-7 cm (or 1-5 cm) from the inlet end of the prosthetic heart valve.
Example 108. The delivery assembly of any example herein, particularly any one of examples 103-107, wherein the second pressure sensor is configured to take measurements within a range of 0-7 cm (or 1-5 cm) from the outlet end of the prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery assembly for a prosthetic heart valve, comprising:
a delivery apparatus;
a guidewire extending through the delivery apparatus;
a first pressure sensor configured to be positioned near an inlet end of a prosthetic heart valve; and
a second pressure sensor configured to be positioned near an outlet end of the prosthetic heart valve,
wherein the first pressure sensor and second pressure sensor are positioned on the delivery apparatus or the guidewire and are configured to measure a pressure gradient across the prosthetic heart valve.

2. The delivery assembly of claim 1, wherein the first pressure sensor and the second pressure sensor are positioned on the guidewire.

3. The delivery assembly of claim 2, wherein at least one of the first pressure sensor and the second pressure sensor are inset in the guidewire.

4. The delivery assembly of claim 1, wherein the first pressure sensor is positioned on the guidewire, and wherein the second pressure sensor is positioned on the delivery apparatus.

5. The delivery assembly of claim 1, wherein the first pressure sensor is positioned on the delivery apparatus, and wherein the second pressure sensor is positioned on the guidewire.

6. The delivery assembly of claim 1, wherein the first pressure sensor and the second pressure sensor are positioned on the delivery apparatus.

7. The delivery assembly of claim 1, wherein the delivery apparatus further comprises an outer sheath, a nosecone, and a nosecone shaft.

8. The delivery assembly of claim 7, wherein the first pressure sensor is positioned on the nosecone shaft and the second pressure sensor is positioned on the guidewire.

9. The delivery assembly of claim 7, wherein the first pressure sensor is positioned on the nosecone, and wherein the second pressure sensor is positioned on the guidewire.

10. The delivery assembly of claim 7, wherein the first pressure sensor is positioned on the nosecone, and wherein the second pressure sensor is positioned on the nosecone shaft.

11. The delivery assembly of claim 7, wherein the first pressure sensor is positioned on the nosecone shaft, and wherein the second pressure sensor is positioned on the guidewire.

12. The delivery assembly of claim 1, wherein the delivery assembly further comprises a fiberoptic cable or an electrical wire for transmitting data from the first pressure sensor and the second pressure sensor.

13. The delivery assembly of claim 1, wherein the delivery assembly further comprises a wireless device for transmitting data from the first pressure sensor and the second pressure sensor and a wireless receiver to receive the transmitted data.

14. The delivery assembly of claim 1, wherein the delivery assembly comprises more than two pressure sensors.

15. The delivery assembly of claim 1, wherein the first pressure sensor and the second pressure sensor are independently movable relative to each other.

16. The delivery assembly of claim 1, wherein the delivery assembly further comprises one or more radiopaque markers.

17. The delivery assembly of claim 1, wherein the delivery assembly further comprises a display apparatus configured to display measurements taken by at least the first pressure sensor and the second pressure sensor.

18. A delivery assembly for a prosthetic heart valve, comprising:
a delivery apparatus;
a first pressure sensor configured to be positioned near an inlet end of a prosthetic heart valve; and
a second pressure sensor configured to be positioned near an outlet end of the prosthetic heart valve,
wherein the first pressure sensor and second pressure sensor are positioned on the delivery apparatus and are configured to measure a pressure gradient across the prosthetic heart valve.

19. The delivery assembly of claim 18, wherein the delivery apparatus comprises a nosecone, a delivery sheath, and a nosecone shaft.

20. A delivery assembly for a prosthetic heart valve, comprising:
a guidewire;
a first pressure sensor configured to be positioned near an inlet end of a prosthetic heart valve; and
a second pressure sensor configured to be positioned near an outlet end of the prosthetic heart valve,
wherein the first pressure sensor and second pressure sensor are positioned on the guidewire and are configured to measure a pressure gradient across the prosthetic heart valve.
